(19) United States
(12) Patent Application Publication
(10) Pub. No.: US 2003/0206920 A1
(43) Pub. Date: Nov. 6, 2003

(54) Proteinic product, process for its preparation, compositions containing it and use in medicaments

(76) Inventor: Rafael Chacon Pabon, Madrid (ES)

Correspondence Address: MICHAELSON AND WALLACE, PARKWAY 109 OFFICE CENTER, 328 NEWMAN SPRINGS RD, P O BOX 8489, RED BANK, NJ, 07701

(21) Appl. No.: 10/138,042

(22) Filed: May 2, 2002

(51) Int. Cl. C12P 21/04; A61K 39/02; C07K 14/32

(52) U.S. Cl. 424/190.1; 530/350; 435/71.2

(57) ABSTRACT

A protein product extracted from apathogen bacilli is described. It comprises proteins, peptides and other molecules, obtained from cellular lysis of at least one of the sporulated and thermal resistant, natural or modified, bacilli strains selected from:

a first group composed of strains of B. Licheniformis, B. Circulans 2, B. Pumilus, B. Macerans, B. Amilolicuofaciens;

a second group composed of strains of B. Cereus 1, B. Cereus 2, B. Lentus 1, B. Lentus 2;

a third group composed of strains of B. Subtilis;

a fourth group composed of strains of B. Mesentericus.

Also is described a process for the preparation of the protein product, compositions containing it and its use in the preparation of medicaments for the treatment of diseases related to immunedeficiency syndrome, auto-immunity, neoplasic processes, degenerative articular diseases and inflammatory intestinal diseases.

### TECHNICAL FIELD OF THE INVENTION

The present invention fits in the field of drugs used for the prevention and treatment of diseases related to immune-deficiency syndrome, neoplasic processes, degenerative diseases, inflammatory intestinal diseases and infectious diseases. More specifically, the invention fits in the sector of drugs obtained from protein extracts of microorganisms.

### PRIOR ART OF THE INVENTION

The mechanisms of diseases related to immunedeficiency syndromes, auto-immunity, neoplasic processes, degenerative diseases, infectious diseases and intestinal inflamations are complex and up to now, there are no drugs that permit the prevention and/or treatment of the syndromes related to the typical clinical patterns of these diseases with a generalized degree of effectiveness for the ill individuals, and that simultaneously have a low enough toxicity so that their administration is tolerated without side effects for the treated individual.

Lately, AIDS symptoms have been treated with encouraging success by means of combinations of drugs. However, one substantial part of the treated individuals do not favorably respond to these drug “cocktails” that, aside from being extremely expensive and require a strict administration program with very frequent doses and they produce adverse side effects in the patients.

There is intense research based on the creation of artificial molecules and the isolation of molecules of natural products, to produce drugs that may fight diseases that are difficult or impossible to cure, such as many of the above mentioned ones. Basically, research has been directed towards finding molecules that inhibit or block certain intracellular and/or metabolic mechanisms that are considered responsible for the diseases, such as neoplasic diseases and diseases related to the immune-system.

Other types of treatments, such as treatments with interferon, that were considered as very encouraging at that moment but criticized in some sectors lately, are the ones that fight the diseases by strengthening the patient's immune-system.

Spanish patents ES-A-348986, ES-A-410892 and ES-A-414437 describe “cancer vaccines” and the process for the preparation thereof, based on extracts obtained by means of bacteriolysis of non-sporulated and non-thermal resistant bacilli strains whose lysates show a positive relationship regarding the specificity against serum of patients afflicted by neoplasias. The “vaccines” described in these Spanish patents, although they were used successfully in a large amount of clinical studies, were relatively complicated to prepare in view of the fact that they were prepared in function of each patient to be treated.

### OBJECT OF THE INVENTION

An object of the present invention is to make available a drug for the prevention and treatment of diseases related to immune-deficiency syndromes, autoimmune diseases, neoplasic and degenerative processes, infectious and infectious intestinal diseases, particularly for the prevention and treatment of individuals afflicted by disorders, such as for example:

immune-deficiency syndromes, among which are those caused by AIDS and idiopathic T CD4 lymphocytopenia;

auto-immunity syndromes, such as those caused by multiple sclerosis, systemic Lupus erythematosus, rheumatoid arthritis, rheumatoid spondylitis, psoriasis and other syndromes with a similar eriopathogenesis;

neoplasic processes, such as those caused by leukemia, myeloma, lymphomas, brain tumors and medulla spinalis tumors, skin cancer, thyroid neoplasia, adrenal gland neoplasia, male and female urogenital system tumors, heart tumors, gastrointestinal tract tumors, pleura and mediastinum tumors, bone and cartilage neoplasia;

syndromes related to degenerative diseases, such as arthrosis;

inflammatory intestinal diseases such as Crohn's disease;

all types of hepatitis; and

diseases caused by prions, such as sub-acute spongiform encephalopathy (Creutzfeldt-Jakob's disease), having in turn a high degree of effectiveness and a practical absence of harmful side effects for the treated individual.

Another object of the invention is to make available a drug with the above described qualities that may also be obtained in a simple and economic manner.

A subsequant object of the invention is to make available a process for the preparation of a drug with the above described qualities, whose process turns out to be sufficiently simple and standardizable, so that it may be carried out in economic and massive conditions.

### DESCRIPTION OF THE INVENTION

The above mentioned objects are achieved by means of a protein product that comprises proteins, peptides and other molecules, obtained from cellular lysis of at least one of the sporulated and thermal resistant bacilli strains, selected from at least one of the following groups:

a first group comprised of strains of B. Licheniformis, B. Circulans 2, B. Pumilus, B. Macerans, B. Amilolicuofaciens, and modified strains thereof;

a second group comprised of strains of B. Cereus 1, B. Cereus 2, B. Lentus 1, B. Lentus 2 and modified strains thereof;

a third group comprised of strains of B. Subtilis and modified strains thereof;

a fourth group comprised of strains of B. Mesentericus and modified strains thereof.

In one embodiment of the invention, the product according to the invention comprises

a least a first fraction of proteins, peptides and molecules obtained from cellular fractionation, selected from among extracts of B. Licheniformis, B. Circulans 2, B. Pumilus, B. Macerans, B. Amilolicuofaciens;

at least a second fraction of proteins, peptides and molecules obtained from cellular fractionation, selected from among extracts of B. Cereus 1, B. Cereus 2, B. Lentus 1, B. Lentus 2;

at least a third fraction of proteins, peptides and molecules obtained from cellular fractionation, selected from among extracts of B. Subtilis;

at least a fourth fraction of proteins, peptides and molecules obtained from cellular fractionation, selected from among extracts of B. Mesentericus.

In another embodiment of the invention, the product according to the invention comprises

at least one first extract that contains proteins, peptides and molecules, obtained from cellular fractionation from bacilli strains of the genus Subtilis

at least one second extract that contains proteins, peptides and molecules, obtained from cellular fractionation of bacilli strains of the genus Cereus;

at least one third extract that contains proteins, peptides and molecules, obtained from bacilli strains of the genus Mesentericus;

at least one fourth extract that contains proteins, peptides and molecules, obtained from bacilli strains of the genus Licheniformis;

at least one fifth extract that contains proteins, peptides and molecules, obtained from bacilli strains of the genus Lentus;

at least one sixth extract thar contains proteins, peptides and molecules, obtained from bacilli strains of the genus Pumilus.

The above mentioned strains are selected from among natural strains and modified strains.

When modified strains are used

the bacilli strains of the first group may be modified strains CECT FCM-1 4913;

the bacilli strains of the second group may be modified strains CECT FCM-2 4914;

the bacilli strains of the third group may be modified strains CECT FCM-3 4915;

the bacilli strains of the fourth group may be modified strains CECT FCM-4 4916.

The above mentioned strains have been deposited in accordance with the provisions of the Budapest Treaty in the “Colección Española de Cultivos Tipo (CECT)=Spanish Culture Type Collection” in the Department of Microbiology of the School of Biology of the University of Valencia, on Jun. 25, 1997.

Hence, the modified strains

B. Licheniformis, B. Pumilus, B. Macerans, B. Amilolicuofaciens, have been catalogued under deposit number CECT FCM-1 4913;

B. Cereus, B. Lentus have been catalogued under deposit number CECT FCM-2 4914;

B. Subtilis has been catalogued under deposit number CECT FCM-3 4915;

B. Mesentericus has been catalogued under deposit number CECT FCM-4 4916.

The protein product typically has the folliwing physical characteristics:

| | |
|---|---|
| Color | yellow |
| pH | 7.0 ± 0.2 |
| Density approx. | 1-1.01 |
| Molecular weight | 5,000-300,000 daltons |

Another object of the invention is also a process for the preparation of a protein product in accordance with the above mentioned characteristics, that basically comprises the following steps:

At least one sporulated and thermal resistant strain is selected from at least one of the following groups:

a first group comprised of strains of B. Licheniformis, B. Circulans 2, B. Pumilus, B. Macerans, B. Amilolicuofaciens, and modified strains thereof;

a second group comprised of strains of B. Cereus 1, B. Cereus 2, B. Lentus 1, B. Lentus 2 and modified strains thereof;

a third group comprised of strains of B. Subtilis and modified strains thereof;

a fourth group comprised of strains of B. Mesentericus and modified strains thereof.

Then, an inoculation of said strain is added to an aqueous solution of a culture medium, in order to obtain an inoculated culture medium. In one embodiment of the process, the aqueous solution may comprise between 19 and 200 g of a culture medium per liter of distilled water. Preferably, the aqueous solution is adjusted to a pH 7.0±0.2 and sterilized in conditions which are conventional, for example, by means of sterilization in an autoclave at 121° C.±1° C. for 15 to 20 minutes.

The temperature of the incoulated culture medium is adjusted in terms of optimal growth of the strain. Preferably, the temperature of the inoculated culture medium is adjusted to a temperature between 18 and 40° C. and forced ventilation is applied by filtered air to the inoculated culture medium during the entire growth stage of the strain, subjecting the inoculated culture medium to an air pressure from 0.5 to 1 l/min.

The strain is cultured in order to achieve a maximum optical density, obtaining a culture that contains a bacillary mass and, then a protein extract is obtained from the bacillary mass. Said extract is obtained by means of the steps of

separation of the bacillary mass from the culture until suspended bacilli are obtained for example by means of cellular concentration of the bacillary mass in the culture by means of centrifugation, cyclone or other processes which are conventional. Preferably, an aliquot part is separated and analyzed before the cellular concentration of the bacillary mass in order to obtain “suspended bacilli” and also to obtain indicative data of the degree of cellular concentration for the purpose of being able to control bacillary growth;

crossed filtration on a filter with a pore diameter smaller than 0.22 micra until a concentrated culture with a concentration range of 10 to 20% is obtained.

Afterwards, the concentrated culture is subjected to diafiltration with physiological saline solution exchange. In a preferred embodiment of the process the concentrated culture is subjected to diafiltration with a membrane filter with a pore size equal to or smaller than 0.22 micra.

Then, an extract that comprises proteins, peptides and molecules that form the active principle is prepared by means of the stages of

subjecting the concentrated culture to cellular lysis, by means of a cryothermal process that comprises a cycle with a first stage at temperatures between 2 and 8° C. for several hours and a second cold stage at a temperature lower than −40° C. for at least 8 hours, preferably at −40 and −50° C. for 8 to 12 hours, and a third stage in a water bath at temperatures between room temperature and 70° C., preferably at a temperature between 60 and 70° C., and more preferably at about 65° C., and optionally, a fourth stage in which the thawed product is kept standing for at least five minutes. Said cycle is repeated at least twice, preferably 4 times, until a cellular lysated concentration that contains the cellular residue and the active principle is obtained;

separating the cellular residue from the active principle subjecting the cellular lysated concentrate to cross flow filtration with diafiltration in a membrane with a pore size smaller than or equal to 0.22 micra with a dilution range of 1:10, in order to obtain an active principle that comprises proteins, peptides and molecules coming from cellular lysis;

concentrating the filtrate using a filter with a pore size such that the proteins with a molecular weight higher than 5000 daltons are retained above the filter in an aqueous solution;

sterile filtration of the concentrated filtrate through a filter with a pore size equal to or smaller than 0.22 micra until a first sterilized protein extract that contains the active principle is obtained.

In a preferred embodiment of the process of the invention, the first sterilized protein extract is mixed with at least one second protein extract prepared similarly to the first extract.

Hence, in a preferred embodiment of the process, the first sterilized protein extract is obtained from at least one strain of said first group and the second sterilized potein extract is obtained from at least one strain of said second group.

According to another preferred embodiment of the process, the first sterilized protein extract and the second sterilized protein extract are also mixed with a third sterilized protein extract obtained from at least one strain of said third group, said third sterilized protein extract being prepared by processes similar to those of the preparation process of the first extract.

In another even more preferred embodiment of the process according to the invention the first sterilized protein extract, the second sterilized protein extract and the third sterilized protein extract are also mixed with a fourth sterilized protein extract obtained from at least one strain of said fourth group, said fourth sterilized protein extract being prepared by processes similar to those of the preparation process of the first extract.

In the above described preferred embodiments, the first sterilized protein extract is preferably obtained from strains CECT FCM-1 4913, the second sterilized protein extract is obtained from strains CECT FCM-2 4914, the third sterilized protein extract is obtained from strains CECT FCM-3 4915, and the fourth sterlized protein estracts is obtained from strains CECT FCM-4 4916.

In order to obtain a “multiuse” drug, in other words, with beneficial effects to prevent and/or treat a plurality of the above described syndromes, the sterilized protein extracts are mixed, in equal proportions, for example 1:1 when two extracts are mixed, 1:1:1 when three extracts are mixed, and 1:1:1:1 when four extracts are mixed, based on their respective protein concentrations. Clearly, when it is desired to strengthen the effects for just one or some of the above described pharmaceutical uses, the different extracts may be mixed in unequal proportions, or just one of the extracts may be used.

The invention also refers to compositions that contain the protein product, comprised of one or more of the above cited protein extracts, as the active ingredient, with a pharmaceutically acceptable vehicle.

Examples of such pharmaceutical compositions are solid compositions, such as tablets, pills, capsules, granules, or liquids, such as lyophilized solutions, suspensions and emulsions, for oral, topical, parenteral administration or by other ways of administration. The doses of these compositions depend on the formulation thereof, the method of administration as well as the place of administration and the individual to be treated. Other factors such as age, body weight, sex, diet, duration of the administration, excretion rate, condition of the individual to be treated, combination with other drugs, the individual's sensibilities and the degree of severity of the disease must also be taken into account. Administration may be done continuously or periodically within the maximum tolerated doses.

When the composition according to the invention is an injectable solution, the liquid medium is, for example, distilled water, the solution being formulated in the following manner:

1 to 99% w/v of the protein product

0 to 99% v/v of distilled water

0.02% v/v of phenol

0-0.12% of pharmaceutically grade formol at 37%

Embodiments of these solutions and of their preparation are specified in the examples.

The present invention is especially applicable in the treatment of HIV positive patients. Nowadays, it is considered that the immunosuppressive effect of HIV can be attributed to the destruction of lymphocytes CD4 that are considered to play a very relevant role in the individual's immune response. There are indications so as to think that the protein products of the present invention stimulate the proliferation of lymphocytes CD4.

Feasible doses to treat AIDS patients are, for example, 1.0, or preferably 2.0 units in 2 ml of water for injections, administered every two days intramuscularly.

It has been possible to verify that in patients afflicted by AIDS to which doses of 2.0 units of a protein product according to the invention were injected intramuscularly, they experiments increases of lymphopcytes CD4 (and also of lymphocytes CD8), without any relevant harmful side effects having been produced.

### BRIEF DESCRIPTION OF THE FIGURES

In order to show some aspects of the invention, in the detailed description of the invention reference will be made to some attached figures, wherein

FIG. 1 shows the results of polyacrylamide electrophoresis analysis (PAGE), of a protein product in accordance with the invention.

FIG. 2 shows the results of some Western Blot analysis to which samples of the protein product according to the invention were subjected.

FIG. 3 summarizes the results of some tests regarding the in vivo response of T cell dependent antibodies.

FIG. 4 summarizes the results of some first tests regarding in vitro activation of B cells.

FIG. 5 is the control of the first tests regarding in vitro activation of B cells in the presence of lipopolysaccharides (LPS).

FIG. 6 summarizes the results of some tests regarding in vitro activation of T cells.

FIGS. 7 to 11 summarize the results of several tests regarding T cell blastogenesis.

### DETAILED DESCRIPTION OF THE INVENTION

The invention will be illustrated hereinafter based on some non-restrictive examples.

### EXAMPLE 1

Material and Equipment

In order to obtain a protein product according to the invention, the following bacilli strains were selected:

B. Licheniformis CECT FCM-1 4913

B. Pumilus CECT FCM-1 4913

B. Circulans CECT-FCM-1 4913

B. Amilolicuofaciens CECT FCM-1 4913

B. Lentus CECT FCM-2 4914

B. Cereus CECT FCM-2 4914

B. Subtilis CECT FCM-3 4915

B. Mesentericus CECT FCM-4 4916

and were injected in four different vials, namely, a first vial with the two strains CECT FCM-1 4913, a second vial with the two strains CECT FCM-2 4914, a third vial with the strain CECT FCM-3 4915, and a fourth vial with the strains CECT FCM-4 4916.

The following equipment and materials were used:

a conventional bacteriological incubator with a stirring device and temperature adjustment (36±1° C.);

water bath thermal vessels;

conventional PROSTAK cross flow filtration equipment with filtration capacities of 40 to 800 l;

conventional unidirectional filtration equipment with a filtration capacity of volumes up to 100 l;

conventional fermentators for the microorganism culture with fermentation capacities for 8.5 l and 800 l;

refrigeration equipment for temperatures from 4 to 8° C.;

freezer equipment for temperatures of −70° C.;

flasks for stirring the cultures with capacities of 2 to 4 l;

wide-mouthed glass vessels with capacities of 5 to 20 l;

PVDF membranes with a pore size of 0.22 micra;

stainless steel containers with a capacity of 50 l, 100 l and 200 l;

culture medium CM-P marketed by INQUIROMA;

water for injection (European Pharmacopoeia=EP)

Bacteriological quality NaCl (EP);

ultrapure phenol (EP);

aqueous solution of formaldehyde of a pharmaceutical grade at 37% (EP)

STRUKTOL™ J 660 antifoam agent.

Primary Strain Propagation (PSP Stage)

2 liters of a first culture solution were prepared, dissolving 35 g of the culture medium CM-P per liter of water, and four flasks were filled with 500 ml of culture solution each. Each one of the solutions were sterilized at 121° C. for 15 min. without having to adjust the pH.

In the first culture solution strains CECTT FCM-1 4913, in the second solution strains CECT FCM-2 4914, in the third culture solution strain CECT FCM-3 and in the fourth solution strain CECT FCM-4 4916 were inoculated. The flask vessels were introduced with the inoculated culture solutions in an incubator and incubations were carried out at 36±1° C. and constant stirring at 100 rpm until a bacillary suspension with an optical density at 620 nm of:

| Group of strains | DO_{620 nm} | time (h)* |
|---|---|---|
| CECT FCM-1 4913 | 4 | 10 |
| CECT FCM-2 4914 | 8 | 15 |
| CECT FCM-3 4915 | 7 | 11 |
| CECT FCM-4 4916 | 4 | 12 |

*= culture termination time

was obtained.

The flasks were removed and each one of the flasks was labeled with the initials PPS followed by the date of preparation and the name of the inoculated strain.

Secondary Strain Propagation (=SSP Stage)

32 liters of a second culture solution were prepared dissolving 35 g of culture medium CM-P per liter of distilled water, adjusting the pH to 7±0.2 with H₃PO₄. The second solution was poured in equal proportions in each one of the four fermentators with a capacity of 8.5 l. 1 ml of STRUKTOL^{TN} J 660 was added to each one of the fermentators.

Steriliation of the second culture solution was carried out for 15 min. at a temperature of 121° C.

Each one of the bacillary suspensions obtained in the PPS stage was transferred respectively to one of the four fermentators with the second sterilized culture solution, so that

the first fermentator contained a culture with 8 l of the second culture solution and 500 ml of bacillary suspension CECT FCM-1 4913;

the second fermentator contained a culture with 8 l of the second culture solution and 500 ml of bacillary suspension CECT FCM-2 4914;

the third fermentator contained a culture with 8 l of the second culture solution and 500 ml of bacillary suspension CECT FCM-3 4915

the fourth fermentator contained a culture with 8 l of the second culture solution and 500 ml of bacillary suspension CECT FCM-4 4916.

The cultures were carried out in their respective fermentators at a temperature of 36±1° C., stirring of the culture medium of 500±100 rpm, with an air flow of 0.8 l/min. and initial dissolved oxygen equal to 98±2% (during the culture >0%), until an optical density at 620 nm of

| Group of strains | DO_{620 nm} | time (h)* |
|---|---|---|
| CECT FCM-1 4913 | 4.4 | 6 |
| CECT FCM-2 4914 | 11 | 10 |
| CECT FCM-3 4915 | 8.9 | 9 |
| CECT FCM-4 4916 | 5.4 | 8 |

*= culture termination time

was obtained.

Main Fermentation Stage (MF Stage)

800 liters of a third culture solution were prepared in a fermentator dissolving 35 g of culture medium CM-P per liter of water, adding 100 ml of Struktol™ J660, adjusting the pH 7±0.2 with 2M H₃PO₄, and sterilizing it at 121° C. for 15 minutes. The third solution was inoculated with the second culture solution and 8.5 l of bacillary solution CECT FCM-1 4913 obtained in the SSP stage, and then fermentation was carried out. Subsequently and successively, respective batches of third solutions were prepared, inoculated and fermented with the strains CECT FCM-2 4914, CECT FCM-3 4915 and CECT FCM-4 4916, in such a way that

8.5 l of SSP culture with strains CECT FCM-1 4913 in 800 l of the third culture solution;

8.5 l of SSP culture with strains CECT FCM-2 4914 in 800 l of the third culture solution;

8.5 l of SSP culture with strains CECT FCM-3 4915 in 800 l of the third culture solution;

8.5 l of SSP culture with strains CECT FCM-4 4916 in 800 l of the third culture solution were fermented.

Each one of the above mentioned cultures were fermented at 36±1° C., with stirring at 300 rpm, a sterile air flow of 0.8 l/min. pH 7±0.2 and initial dissolved oxygen 98±2% (during the culture >0%), until a stationary growth stage was reached in each culture until an optical density at 620 nm of

| Group of strains | DO_{620 nm} | time (h)* |
|---|---|---|
| CECT FCM-1 4913 | 5 | 10 |
| CECT FCM-2 4914 | 27 | 14 |
| CECT FCM-3 4915 | 27 | 13 |
| CECT FCM-4 4916 | 6 | 12 |

*= culture termination time

was obtained.

Harvest

The four cultures obtained by means of the MF stage were transferred in batches to different containers of 50 l, labeling each one of them with the initials MF, the preparation date thereof and the name of the cultured strain.

Preparation of a Concentrated Cellular Suspension (CCS Stage)

Each one of the harvested cultures was filtered in cross flow in PROSTAK filtration equipment with a PVDF membrane of 0.22 micra, until respective concentrations 20 times more concentrated than the respective harvested cultures, were obtained. Subsequently, each one of the concentrated cultures was subjected to diafiltration with physiological saline solution exchange in PROSTAK equipment with a membrane filter with 0.22 micra with 150 l of physiological saline solution, 10 l of each one of the concentrated cellular suspensions was transferred to containers with a 20 l capacity, labeling each container with the initials CCS, date of preparation thereof and name of the cultured strain.

Obtainment of Lysated Cell Suspensions by Freezing/Heating

Each group of containers with concentrated cellular suspensions resulting from the CCS step with the same strain, was subjected to a cryothermal process repeating the following cycle four times:

refrigerating to 4° C. for 3 hours in refrigerating equipment;

freezing to −40° C. for 18 hours in freezing equipment;

thawing putting in heating equipment with hot water at 65° C. until complete thawing.

At the end of each cycle a count of CFU and total protein content (Kjeldahl) in each one of the lysated cell suspensions was carried, obtaining the following values:

| Cycle | | | |
|---|---|---|---|
| (g/l) | contained strain | CFU/ml | total protein |
| before cycle | CECT FCM-1 4913 | 1 × 10⁸ | 11.1 |
| 1 | | <10⁴ | 11.8 |
| 2 | | 2 × 10⁶ | 10.5 |
| 3 | | 2 × 10⁶ | 10.5 |
| 4 | | 2 × 20⁶ | 10.5 |
| before cycle | CECT FCM-2 4914 | 5 × 10⁹ | 9.4 |
| 1 | | 1 × 10⁸ | 20.5 |
| 2 | | 5 × 10⁶ | 18.3 |
| 3 | | 1 × 10⁴ | 18.8 |
| 4 | | 2 × 10⁴ | 20.8 |
| before cycle | CECT FCM-2 4915 | 3 × 10¹⁰ | 14.7 |
| 1 | | 1 × 10⁴ | 16.8 |
| 2 | | 1 × 10⁴ | 17.0 |
| 3 | | 1 × 10⁴ | 17.0 |
| 4 | | 1 × 10⁴ | 17.0 |
| before cycle | CECT FCM-2 4916 | 6 × 10⁷ | 8.3 |
| 1 | | 2 × 10⁶ | 8.6 |
| 2 | | 2 × 10⁶ | 9.7 |
| 3 | | 2 × 10⁶ | 9.1 |
| 4 | | 1 × 10⁶ | 9.6 |

Obtainment of Protein Extracts with the Active Ingredient Free of Cellular Detritus (AI Stage)

(a) Obtainment of Filtered Protein Extracts (AIP Stage)

The lysated cell suspensions, grouped respectively according to the strains of origin, resulting from the cryothermal process were subjected to cross filtration in PROSTAK equipment with a membrane filter of 0.22 micra.

In order to know the amount of cellular detritus, respective samples were taken at the end of each cross filtration process and the respective DO₆₂₀ values were determined before and after centrifugation at 4,000 rpm for 15 minutes. The absorbency difference in each sample was less than 0.1 and, therefore, the filtered protein extracts had acceptable qualities. Each one of the extracts resulting from the AIP stage was labeled with the initial AIP, the date of preparation thereof, and the name of the strain of origin.

(b) Diafiltration with Physiological Saline Solution Exchange (AIP)

Each one of the protein extracts resulting from the AIP stage was subjected to diafiltration in PROSTAK equipment with a membrane filter of 0.22 micra with a physiological saline solution volume (0.9% m/v) ten times greater than the volume of filtered protein extracts, controlling the diafiltration by analysis of the total protein content (Kjeldahl) in the filtrate, until an optimum protein content of:

| Contained strain | Total protein (g/l) |
|---|---|
| CECT FCM-1 4913 | 1.3 |
| CECT FCM-2 4914 | 5.9 |
| CECT FCM-3 4915 | 6.9 |
| CECT FCM-4 4916 | 2.5 |

has been achieved.

Each one of the extracts resulting from the AIP stage (approximately 40 l per extract) was introduced into a container that was labeled with the initials AIP, the date of preparation thereof, and the name of the strain of origin.

Obtainment of the Concentrated Active Ingredient (CAI Stage)

Each one of the extracts resulting from the AIP stage was subjected to concentration by means of cross flow filtration equipment in PROSTAK equipment with a membrane filter of 5000 daltons of a nominal pore diameter (Pellicon, Biomax 5k, Polyethersulfon) in order to obtain a concentration 1:2, in other words, a volume of approximately 20 l of each active ingredient resulting from the CAI stage.

Samples were taken of each active ingredient CAI in order to determine their protein content (Kjeldahl). The analysis of these samples was carried out in order to have a control to check that the process is being carried out correctly and, therefore, is basically similar to the verification in the AIP stage.

The concentrated active ingredients resulting from the CAI stage were introduced in respective containers labeled with the initials CAI, the date of preparation thereof, and the name of strain of origin.

Obtainment of the Sterile Active Principle (SAI Stage)

Each one of the concentrated active ingredients resulting from the CAI stage was subjected to filtration in unidirectional equipment with a PVDF membrane with a pore size of 0.22 micra. A sample was taken of each sterilized principle for a sterility test after filtration. The criteria of acceptance is the retention of microorganisms in the filter is less than 107/cm². In the present case, the following values

| | Before | Filter | |
|---|---|---|---|
| | Filtering | retention | After |
| Contained strain | CFU/cm² | CFU/cm² | filtering |
| CECT FCM-1 4913 | <10 | <110 | sterile |
| CECT FCM-2 4914 | 40 | 7 × 10² | sterile |
| CECT FCM-3 4915 | 60 | 4 × 10² | sterile |
| CECT FCM-4 4916 | <10 | <110 | sterile |

was obtained.

The protein concentration (Kjeldahl) was also analyzed in order to keep a control of the process and to be able to check that the process is being carried out correctly. For this purpose, a sample of the solution is taken and the protein concentration is measured. This step is similar to the verification in the AIP stage.

The resulting active principles were introduced in a Nalgene container labeled with the initials SAI, the date of preparation and the name of the strain of origin and were stored in a refrigerator at 4° C.

Preparation of the Protein Product in Bulk (B Stage)

100 l of water for injection were put in a suitable vessel and, under constant stirring, phenol at a concentration of 0.2% (m/v), with respect to a volume of 200 l, formaldehyde solution at a concentration of 0.02% (v/v), also with respect to a volume of 200 l, and sterilized active principles coming from the SAI stage, in a ratio of SAI CECT FCM-1 4913: SAI CECT FCM-2 4914: SAI CECT FCM-3 4915: SAI CECT FCM-4, of 1:1:1:1, with reference to their respective protein concentrations, were added. To the mixture thus obtained, water for injection was added until completing a volume of approximately 200 l and the resulting mixture was kept under stirring for 30 minutes, obtaining the crude product in bulk.

The product in bulk was subjected to sterile filtration using unidirectional filtration equipment with a coupled PVDF membrane, of 0.22 micra, obtaining a sterile product in bulk. A sample was subjected to sterility and protein concentration (Kjeldahl) tests, following the systems and criteria used in the sterility tests and protein analyses of the previous steps.

The sterile product in bulk was packaged in a Nalgene container labeled with the initial B, the date of preparation and the names of the strains of origin, and it was stored at 4° C. in a refrigerator.

Preparation of the Product Dosed for Injections

The sterile product in bulk was dispensed in ampoules in the amount of 2 ml per ampoule.

### EXAMPLE 2

Three samples of the product in bulk obtained in example 1 were analyzed to characterize their protein component by means of polyacrylamide gel electrophoresis (PAGE). The analyzed samples did not require prior preparation.

The proteins of each one of the samples were separated by PAGE techniques, in standard BIORAD 10% polyacrylamide tris-glycine, of BIIORAD, USA.

A denaturalizing buffer with 10% SDS and 5% 2-mercaptoethanol was used. The electrode buffer was the buffer Tris-glycine/SDS used conventionally for this type of gel (Laemmli method).

The separation parameters were 125 V, 30 inA (starting conditions) for approximately 90 min.

Then the gels were stained with Coomassie blue and were discolored to show the protein bands, in accordance with the following protocol:

Gel staining solution (at 500 ml)

| | | |
|---|---|---|
| Coomassie blue | 500 | mg |
| Ethanol | 200 | mg |
| Distilled water | 250 | ml |
| Glacial acetic acid | 50 | ml |

Each gel was stained with 100 ml of solution and left to stand for at least 30 minutes.

Gel decoloring solution (at 500 ml)

| | |
|---|---|
| Ethanol | 60 ml |
| Distilled water | 400 ml |
| Glacial acetic acid | 40 ml |

Each gel remained in this solution overnight.

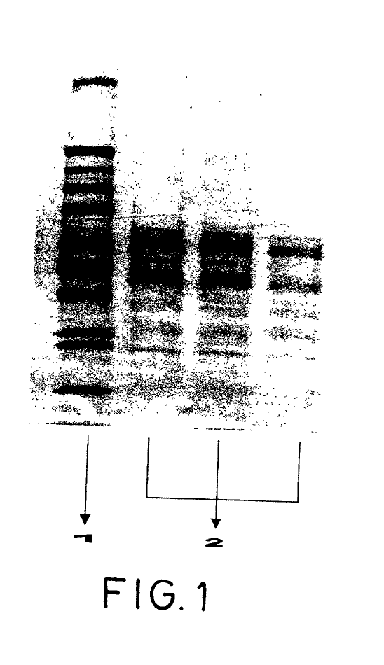

The results of the PAGE test are shown in FIG. 1 in which numbers 1-2 have the following meanings:

1=Sigma Marker No. M-4038 column

2=Protein product columns

As one can see, this figure shows different bands of distinct intensities, but rather band standards practically identical to the three tested products, which is indicative that the samples had the same protein composition.

### EXAMPLE 3

### Two other samples of the product in bulk of example 1 were taken and subjected to polyacrylamide gel electrophoresis (PAGE) in the same conditions as those described in example 1.

Subsequently, the proteins of each sample, separated by PAGE in accordance with their molecular weights, were electrotransferred to a single nitrocellulose membrane in order to permit subsequent analysis. The corresponding methodology is described by Sambrock, Fritsch and Maniatis, Molecular Cloning—A Laboratory Manual, Cold Spring Harbor (1989).

A LINUS electrotransfer camera was used, with LINUS tris-glycine with electrotransfer, at 200 mA, 40 V and for 2 hours.

The membranes on which the proteins separated by PAGE corresponding to each one of the samples were transferred were subjected to immunoassay with polyclonal antibodies produced in rabbits that had been previously obtained by means of treatment with a different batch of the protein product obtained in accordance with the method described in example 1 and that was found in rabbit serum stored at −180° C.

Given that the antigen-antibody reaction is very specific and, therefore, the attachment of the protein product to the bands, each one of which corresponds to a protein of the preparation, produced by each one of the samples is indicative of the presence of common proteins in the preparation used to immunize the rabbits, and, consequently, it may serve for quality control of the new preparations that are produced.

In order to obtain the serum with polyclonal antibodies produced in rabbit of the protein product according to the invention, we proceeded in the following manner:

Two young and healthy New Zealand white albino rabbits, one male and one female, were selected and kept isolated and in observation for 5 days for the acclimation to the environment of the installation. After the acclimation period and without having observed any anomaly in the animals, the following program was followed:

Way of administration: intramuscular injection

Days of administration: 1, 7, 14, 17

The intramuscular administration was carried out by means of disposable syringes with needles with a caliber of 16×0.5 mm and the administration was applied to each animal on the rear surface of the rear leg, alternating the right and lefts sides.

The test was considered ended on day 23 and the animals were sacrificed, after being anesthetized by an intraperitoneal injection of sodium pentothal in an aqueous solution, by exsanguination, removing the blood by cardiac puncture. The blood was collected without any type of additive, and the serum was obtained by low speed centrifugation.

The obtained serum (40 ml) was homogenized, aliquoted in cryotubes of 1 and 4.5 ml, referenced as “serum-97” and frozen at −180° C. in liquid nitrogen.

The methodology used is in itself conventional and corresponds to the ones described in:

Donovan, J. and Brown, P., 1995a, Parenteral injections in current protocols in immunology; 1.6.1-1.6.10; John Wiley & Sons, New York

Current Protocols in Molecular Biology, 1994, Ed. Virginia Benson Chanda

Hurrell J. G. R. Ed. 1982, Monoclonal Hybridoma Antibodies: Techniques and applications, CRC Press, Boca Raton, Fla.

Lagone, J. J. and Van Vunakis, H. H. Eds. 1986, Immunological Techniques, Part I: Hybridoma Technology and Monoclonal Antibodies, Methods Enzymol., 121:1-947

In view of the fact that it is possible to detect primary antibodies, that have been attached to the proteins, using a technique that implies the use of a second marked antibody produced in a different host than that which was used to produce the first antibody, said second antibody produced in goats, sensitive to light and to heavy rabbit IgG chains, and that consequently was capable of detecting any rabbit antibody linked to proteins on the nitrocellulose membrane, was used. Each one of the second antibodies have a marker (PIERCE horse radish peroxidase, Rockford, Ill., United States of America) that permits visualization of the presence of such antibodies on the membrane when a reagent, 4-chloronaphthol, that produces a violet stain, is added.

The second antibody used in the present test was an commercial antibody PIERCE IMMUNOPURE GOAT ANTI-RABBIT IgG (H+L) (product no.: 31460; batch no.: 96060545), Rockford, Ill., United States of America.

Each one of the violet bands on each one of the nitrocellulose membranes corresponds to a specific protein that was also found in the preparation of the control of the protein product that had been used to immunize the two rabbits, from whose serum the first antibody had been extracted.

For the treatment of each one of the membranes, the following compositions were used:

| Solution 1: | | |
|---|---|---|
| PBS (without Ca nor Mg) | 300 | ml |
| Tween 20 | 150 | μ |
| Solution 2: | | |
| solution 1 | 50 | ml |
| BSA | 500 | mg |
| Powdered milk | 2.5 | mg |
| Solution 3: | | |
| solution 1 | 50 | ml |
| BSA | 250 | mg |
| Washing solution: | | |
| PBS (without Ca nor Mg) | 200 | ml |
| Powdered milk | 1 | g |
| Tween 20 | 100 | μl |
| First antibody solution (1/100): | | |
| First antibody | 200 | μl |
| Solution 3 | 20 | ml |
| Second antibody solution (1/1500): | | |
| Second antibody (PIERCE) | 60 | μl |
| Solution 3 | 30 | ml |
| Tris-HCl solution: | | |
| Tris-HCl | 160 | mg |
| NaCl | 820 | mg |
| Distilled water | 100 | ml |
| 4-CN solution: | | |
| 4-chloronaphthol (PIERCE) | 30 | mg |
| Ethanol | 10 | ml |
| Developing solution: | | |
| Tris-HCl solution | 50 | ml |
| 4-CN solution | 5 | ml |
| H₂O₂ | 10% | |

Each nitrocellulose membrane with the transferred proteins was subjected to the following treatment:

Double washing in solution 1 for 1 min.;

Incubation in solution 2 for 8 hours;

Double washing in solution 1 for 30 min.;

Incubation in the solution of the first antibody for 2 hours;

Four washings in the washing solution for 10 min.;

Incubation for 2 hours in the solution of the second antibody;

Four washing in the washing solution for 10 min.;

Developing in the developing solution for 30 min.;

Washing with ultrapure water and kept in darkness until the photographs are taken.

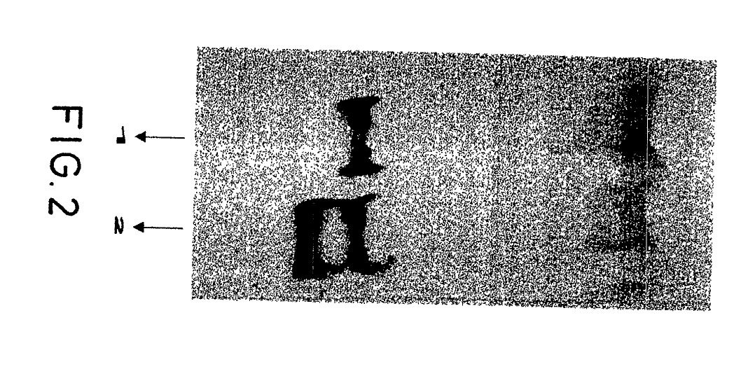

After this treatment, the microcellulose membranes were photographed. The photographs are shown in FIG. 2.

In accordance with what is inferred from FIG. 2, the analyzed samples showed practically identical band standards and band intensities, that is to say, they were homogeneous to each other as to their compositions in proteins and with respect to the rabbit serum.

Biological Activity Tests

Immunostimulation was evaluated on the basis of the tests carried out with samples of the protein product obtained according to example 1. The samples are identified as FR-91.

1) T-Dependent In Vivo Response Using the Platelet Forming Cell Test

Two mice were immunized with 0.025-0.3 ml of FR-91 per mouse.

The mice were sacrificed by means of cervical dislocation and the spleen of each one was removed.

After four days, immunoglobulins conjugated with proteins SRBC with splenic lymphocytes were mixed, among which specific antibody producing cells are found, and were incubated in a semi-solid vehicle medium in order to permit the secreted antibodies to attach to the surfaces of the erythrocytes. Then, a complement with SRBC conjugated antibody specificity was added to the mixture, after which the areas free of lysis (plaque) were measured in order to evaluate the activation of lymphocytes.

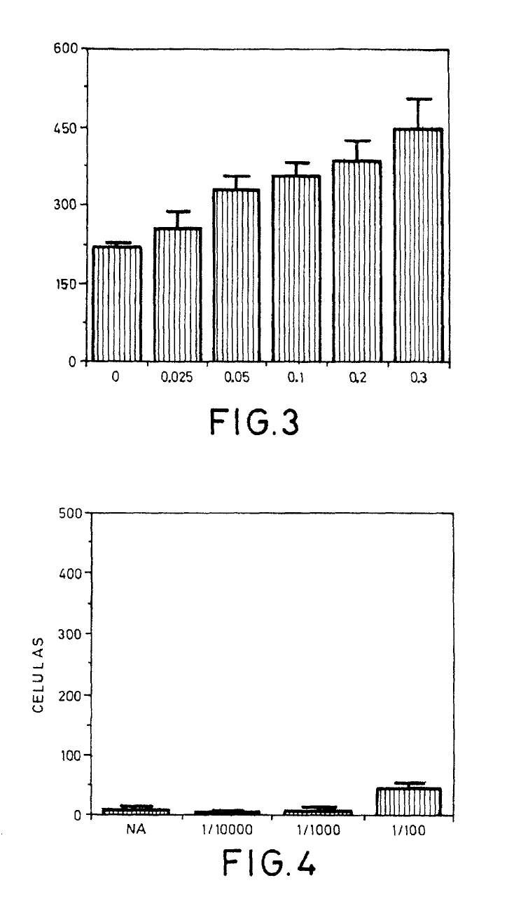

In accordance with what is inferred from FIG. 3, the samples with FR-91 had an increased number of T-dependent antibody producing cells in mice immunized with sheep red blood cells, which is indicative that FR-91 is effective in the immune system related to many types of lymphocytes.

2) B Cell In Vitro Activation, Using the Plaque Forming Cells Test

Samples of FR-91 were added to splenocytes making final concentrations from 100:1 (1%) to 10000:1 (0.01%) and the level of T %-dependent antibody producing cells with or without lipopolusaccharides was measured using a platelet forming cell test.

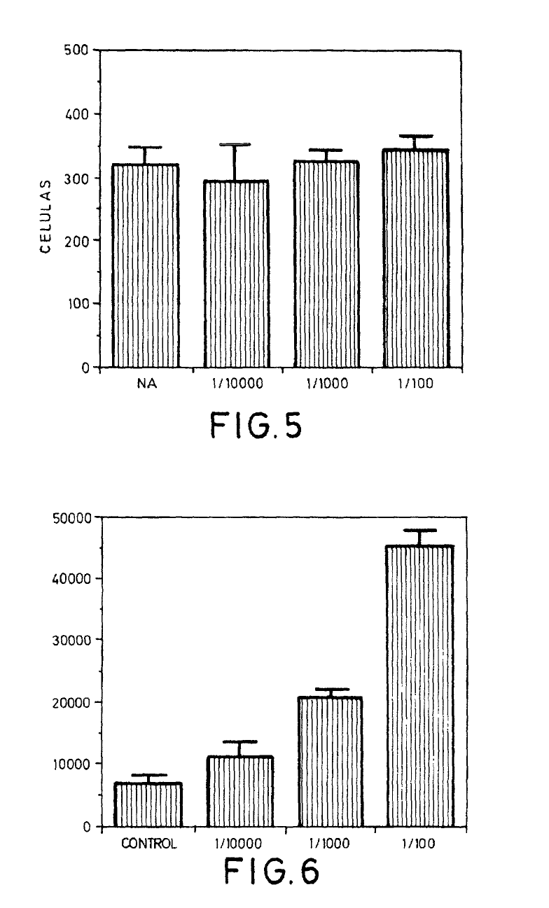

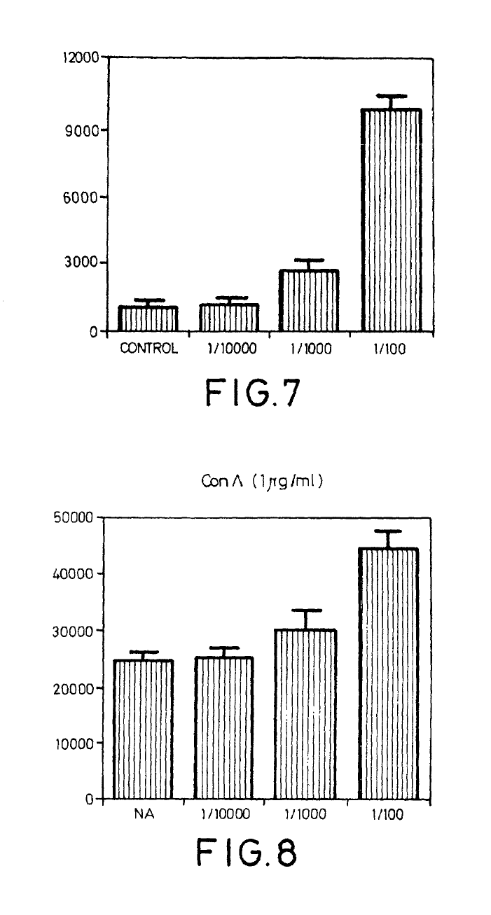

In accordance with what is inferred from FIGS. 4 and 5, the addition of FR-91 practically did not cause any increase in the proliferation or activation of B cells, upon treating splenocytes without or without lipopolysaccharides.

3) T Cell In Vitro Activation

When cultivating together lymphocytes of two different endogamic strains, the cells began to proliferate in response to the antigenic differences in the alogenic lymphocytes. The intensity of this mixed lymphocyte reaction (MLR) may be quantified bu adding thymidine marked with tritium to the culture medium. Given that the cells proliferate, the radioactive thymidine mixes with the DNA of the descendent cells. The degree of proliferation is determined by harvesting the cells, subjecting them to lysis, and measuring the amount of radioactive thymidine included in the DNA which is directly proportional to the level of proliferation.

Based on the above defined methodology, the following test was carried out:

Lymphocytes of two different endogamic strains were mixed in some micropocill.

Series diluted samples of FR-91 in concentrations of 100:1, 1000:1 and 10000:1 were added to the micropocill.

After cultivating for 4 days, tritrated thymidine [³H]was added to the respective mixtures (C₁₀³H₁₄N₂O₅; SIGMA of SIGMA Corp., USA).

After cultivating for 20 hours, the culture cells were harvested in the micropocill on a paper-filter strip, and the thymidine [³H] included in the DNA of the respective samples was measured and compared with the control sample without FR-91.

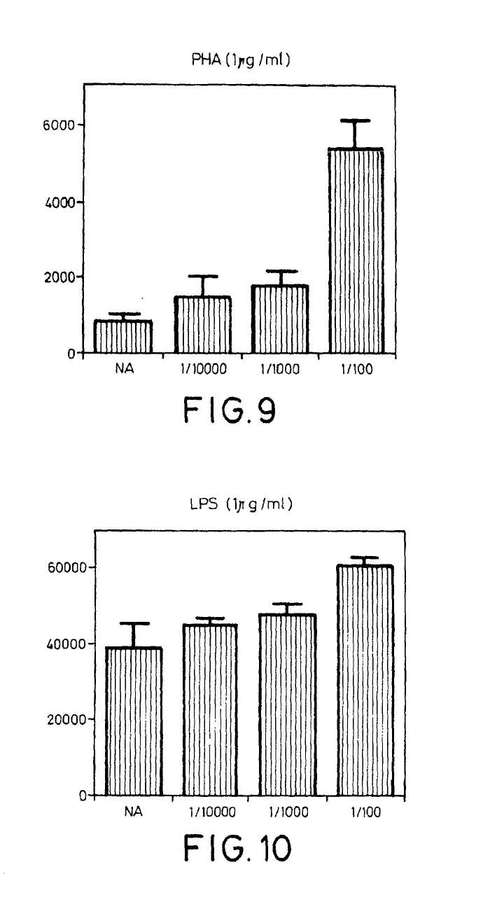

In accordance with what is inferred from FIG. 6, FR-91, strongly increased the proliferation of T cells.

4) T Cells Blastogenesis

Series diluted samples of FR-91 were added to splenocytes in final concentrations of 100:1, 1000:1 and 10,000:1 and were introduced in micropocill.

Respectively identified micropocill were treated with lipopolysaccharides, pokeweed mythogene, phytohematoglutinine, concanavaline A as the standard mythogene, at a final concentration of 1 μg/pocill.

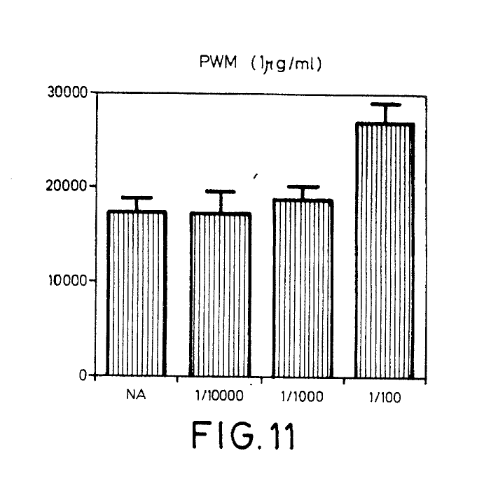

The T and B cell proliferation level was measured by thymidine [³H] addition tests. The results are reflected in FIGS. 7-11.

In accordance with what is inferred from FIG. 7, FR-91 increased intensely the splenocyte immunity without any mythogene.

FIG. 8 shows that FR-91 produces a synergic effect with concanavaline A, which is a T cell mythogene.

FIG. 9 evidences that FR-91 has a strong synergism with phytohematoglutinine, which is a T cell mythogene.

FIG. 10 evidences that FR-91 and lipopolysaccharides, which are mythogenes of B cells, do not have any synergy.

FIG. 11 shows, at a concentration of 100:1, that FR-91 produces some synergic effect with pokeweed mythogene, which s a common T and B mythogene.

From the above it is inferred that FR-91 substantially increases T cell proliferation and activation, but does not have any effects on B cells.

1. Protein product that comprises fractions of peptides, proteins and other molecules, removed from apathogenic bacilli, characterized in that
the product is at least one protein extract and comprises proteins, peptides and other molecules, obtained from cellular lysis of at least one of the sporulated and thermal resistant bacilli strains, selected from at least one of the following groups:
a first group comprised of strains of B. Licheniformis, B. Circulans 2, B. Pumilus, B. Macerans, B. Amilolicuofaciens, and modified strains thereof;
a second group comprised of strains of B. Cereus 1, B. Cereus 2, B. Lentus 1, B. Lentus 2 and modified strains thereof;
a third group comprised of strains of B. Subtilis and modified strains thereof;
a fourth group comprised of strains of B. Mesentericus and modified strains thereof.

2. Protein product according to claim 1, characterized in that it comprises
at least one first fraction of proteins, peptides and molecules obtained from cellular fractionation, selected among extracts of B. Licheniformis, B. Circulans 2, B. Pumilus, B. Macerans, B. Amilolicuofaciens;
at least one second fraction of proteins, peptides and molecules obtained from cellular fractionation, selected from among extracts of B. Cereus 1, B. Cereus 2, B. Lentus 1, B. Lentus 2;
at least one third fraction of proteins, peptides and molecules obtained from cellular fractionation, selected from among extracts of B. Subtilis;
at least one fourth fraction of proteins, peptides and molecules obtained from cellular fractionation, selected from among extracts of B. Mesentericus.

3. Protein product according to claim 1, characterized in that it comprises
at least one first extract that contains proteins, peptides and molecules, obtained from cellular fractionation of bacilli strains of the genus Subtilis;
at least one second extract that contains proteins, peptides and molecules, obtained from cellular fractionation of bacilli strains of the genus Cereus,
at least one third extract that contains proteins, peptides and molecules, obtained from bacilli strains of the genus Mesentericus;
at least one fourth extract that contains proteins, peptides and molecules, obtained from bacilli strains of the genus Licheniformis;
at least one fifth extract that contains proteins, peptides and molecules, obtained from bacilli strains of the genus Lentus;
at least one sixth extract that contains proteins, peptides and molecules, obtained from bacilli strains of the genus Pumilus.

4. Protein product according to claim 1, characterized in that the bacilli strains are selected from among natural strains and modified strains.

5. Protein product according to claim 1, characterized in that the bacilli strains of the first group are modified strains CECT FCM-1 4913.

6. Protein product according to claim 1, characterized in that the bacilli strains of the second group are modified strains CECT FCM-2 4914.

7. Protein product according to claim 1, characterized in that the bacilli strains of the third group are modified strains CECT FCM-3 4915.

8. Protein product according to claim 1, characterized in that the bacilli strains of the fourth group are modified strains CECT FCM-4 4916.

9. Protein product according to claim 2, characterized in that the modified strains of B. Subtilis are strains CECT FCM-3 4915.

10. Protein product according to claim 2, characterized in that the modified strains of B. Cereus are strains CECT FCM-2 4914.

11. Protein product according to claim 2, characterized in that the modified strains of B. Mesentericus are strains CECT FCM-4 4916.

12. Protein product according to claim 2, characterized in that the modified strains of B. Licheniformis are strains CECT FCM-1 4913.

13. Product according to claim 2, characterized in that the modified strains of B. Lentus are strains CECT FCM-2 4914.

14. Product according to claim 2, characterized in that the modified strains of B. Pumilus are strains CECT FCM-1 4913.

15. Process for the preparation of the protein extract, characterized in that it comprises the steps of:
selecting at least one sporulated and thermal resistant strain from at least one of the following groups:
a first group comprised of strains of B. Liceniformis, B. Circulens 2, B. Pumilus, B. Macerans, B. Amilolicuofaciens, and modified strains thereof;
a second group comprised of strains of B. Cereus 1, B. Cereus 2, B. Lentus 1, B. Lentus 2 and modified strains thereof;
a third group comprised of strains of B. Subtilis and modified strains thereof;
a fourth group comprised of strains of B. Mesentericus and modified strains thereof;
adding an inoculation of said strain to an aqueous solution of a culture medium, in order to obtain an inoculated culture medium,
adjusting the temperature of the inoculated culture medium in terms of the optimum growth of the strain;
cultivating the strain until achieving a maximum optical density until obtain a culture that contains a bacillary mass;
obtaining a protein extract of the bacillary masses by means of the steps of:
separating the bacillary mass from the culture until obtaining suspended bacilli; cross filtration on a filter with a pore diameter smaller than 0.22 micra until a concentrated culture with a concentration range of 10 to 20 is obtained;
subjecting the concentrated culture to diafiltration with physiological saline solution exchange;
obtaining an extract that comprises proteins, peptides and molecules that form the active principle by means of the stages of
subjecting the concentrated culture to cellular lysis, by means of a cryothermal process that comprises a cycle with a first stage at temperatures between 2 and 8° C. for several hours and a second cold stage at a temperature lower than −40° C. for at least 8 hours, and a third stage in a water bath at temperatures between room temperature and 70° C.; repeating said cycle at least twice, until a cellular lysate concentrate that contains a cellular residue and the active principle is obtained;
separating the cellular residue from the active principle subjecting the cellular lysate concentrate to cross flow filtration with diafiltration in a membrane with a pore size smaller than or equal to 0.22 micra with a dilution range of 1:10, in order to obtain a crude protein extract filtrate that contains an active principle that comprises proteins, peptides and molecules coming from cellular lysis;
concentrating the filtrate using a filter with a pore size such that the proteins with a molecular weight higher than 5000 daltons are retained above a filter in an aqueous solution;
sterile filtration of the concentrated filtrate through a filter with a pore size equal to or smaller than 0.22 micra until a first sterilized protein extract that contains the active principle is obtained;
optionally, mixing the first sterilized protein extract, with at least a second protein extract prepared similarly to the first extract.

16. Process according to claim 15, characterized in that the aqueous solution comprises between 19 and 200 g of a culture medium per liter of water of microbiological quality.

17. Process according to claim 15, characterized in that the aqueous solution is adjusted to a pH of 7.0±0.2.

18. Process according to claim 15, characterized in that the aqueous solution is sterilized at least at 121° C. for 15 to 20 minutes.

19. Process according to claim 15, characterized in that the temperature of the inoculated culture medium in question is adjusted to a temperature between 18 and 40° C.

20. Process according to claim 15, characterized in that filter air forced ventilation is applied to the inoculated culture medium until the beginning of the strain growth stage subjecting the inoculated culture medium to an air pr essure of 0.5 to 1/ml.

21. Process according to claim 15, characterized in that the concentrated culture is obtained by cellular concentration of the bacillary mass in the culture by means of centrifugation, cyclone or other process which are in themselves conventional.

22. Process according to claim 15, characterized in that before cellular concentration of the bacillary mass an aliquot part of the culture is separated and analyzed before concentration in order to obtain data of “suspended bacilli” in order to obtain indicative data of the degree of concentration.

23. Process according to claim 15, characterized in that the concentrated culture is subjected to diafiltration with a membrane filter with a pore size equal to or smaller than 0.22 micra.

24. Process according to claim 15, characterized in that the second stage of the cycle of the cryothermal process is carried out at −40 and −50° C. for 8 to 12 hours and because the third stage is carried out at a temperature between 60 and 70° C.

25. Process according to claim 15, characterized in that the cryothermal process is comprised of 4 of said cycles.

26. Process according to claim 15, characterized in that the heating to reach the complete thawing of the concentrated culture is done at a temperature lower than 65° C.

27. Process according to claim 15, characterized in that the thawed product is kept standing for at least minutes in a fourth stage of each cycle of the cryothermal process.

28. Process according to claim 15, characterized in that the first sterilized protein extract is obtained from at least one strain of said first group and in that the second sterilized protein extract is obtained from at least one strain of said second group.

29. Process according to claim 28, characterized in that the first sterilized protein extract and the second sterilized protein extract are also mixed with a third protein extract obtained from at least one strain of said third group, said third sterilized protein extract having been prepared by processes similar to those of the preparation process of the first extract.

30. Process according to claim 29, characterized in that the first sterilized protein extract, the second sterilized protein extract and the third sterilized protein extract are also mixed with a fourth sterilized protein extract obtained from at least one strain of said fourth group, said fourth sterilized protein extract having been prepared by processes similar to those of the preparation process of the first extract.

31. Process according to claim 28, characterized in that the first sterilized protein extract is obtained from strains CECT FCM-1 4913, the second sterilized protein extract is obtained from strains CECT FCM-1 4914.

32. Process according to claim 29, characterized in that the first sterilized protein extract is obtained from strains CECT FCM-1 4913, the second sterilized protein extract is obtained from strains CECT FCM-2 4914 and the third sterilized protein extract is obtained from strains CECT FCM-3 4915.

33. Process according to claim 30, characterized in that the first sterilized protein extract is obtained from strains CECT FCM-1 4913, the second sterilized protein extract is obtained from strains CECT FCM-2, the third sterilized protein strain is obtained from strains CECT FCM-3 and the fourth sterilized protein extract is obtained from strains CECT FCM-4 4916.

34. Process according to claim 28, characterized in that the sterilized protein extracts are mixed in equal proportions referred to their respective protein concentrations.

35. Pharmaceutical composition characterized in that it contains a protein product according to claim 1 and a pharmaceutically acceptable vehicle.

36. Pharmaceutical composition characterized in that it contains a protein product according to claim 1, in solution in a liquid medium.

37. Composition according to claim 36, characterized in that the liquid medium is an aqueous solution.

38. Composition according to claim 36, characterized in that the liquid medium is distilled water.

39. Composition according to claim 38, characterized in that it contains
1 to 99% w/v of the protein product
0 to 99% v/v by weight of distilled water
0-0.2% v/v of phenol
0-0.12% v/v pharmaceutically grade formol at 37%.

40. Use of a protein product according to claim 1, in the production of drugs used in the prevention and treatment of disorders in individuals with immune-deficiency syndromes.

41. Use according to claim 40, in the production of drugs for the prevention and treatment of AIDS.

42. Use according to claim 40, in the production of drugs for the prevention and treatment of idiopathic T CD₄+lymphocytopenia.

43. Use of a protein product of claim 1, in the production of drugs used in the prevention and treatment of disorders in individuals with auto-immunity syndromes.

44. Use according to claim 43, in the production of drugs for the prevention and treatment of any of the syndromes related to multiple sclerosis, systemic Lupus erythematosus, rheumatoid arthritis, rheumatoid spondylitis and psoriasis.

45. Use of a protein product according to claim 1, in the production of drugs used in the prevention and treatment of disorders in individuals afflicted by neoplasic processes.

46. Use according to claim 45, in the production of drugs for the prevention and treatment of any of the syndromes related to neoplasic processes selected among leukemia, myeloma, lymphomas, brain tumors and medulla spinalis tumors, skin cancer, thyroid neoplasia, adrenal gland neoplasia, male and female urogenital system tumors, cardiac tumors, gastrointestinal tract tumors, lung, pleura and mediastinum tumors and bone and cartilage neoplasia.

47. Use of a protein product according to claim 1, in the production of drugs used in the prevention and treatment of disorders in individuals with syndromes related to degenerative diseases.

48. Use according to claim 47, in the production of drugs for the prevention and treatment of arthrosis.

49. Use of a protein product according to claim 1, in the production of drugs used in the prevention and treatment of disorders in individuals with syndromes related to inflammatory intestinal diseases.

50. Use according to claim 49, in the production of drugs in the prevention and treatment of Crohn's disease.

51. Use of a protein product according to claim 1, in the production of drugs used in the prevention and treatment of disorders in individuals with syndromes related to hepatitis.

52. Use of a protein product according to claim 1, in the production of drugs used in the prevention and treatment of disorders caused by prions in individuals.

53. Use according to claim 52, in the production of drugs in the prevention and treatment of Creutzfeldt-Jakob's disease.
